(19) United States
(12) Patent Application Publication
(10) Pub. No.: US 2025/0046340 A1
(43) Pub. Date: Feb. 6, 2025

(54) NON-LINEAR OPTICAL-ABSORBING MATERIAL, RECORDING MEDIUM, METHOD FOR RECORDING INFORMATION, AND METHOD FOR READING INFORMATION

(71) Applicant: Panasonic Intellectual Property Management Co., Ltd., Osaka (JP)

(72) Inventors: MASAKO YOKOYAMA, Osaka (JP); KOTA ANDO, Osaka (JP); NAOYA SAKATA, Osaka (JP); KENJI TAGASHIRA, Osaka (JP); HIDEKAZU ARASE, Hyogo (JP)

(21) Appl. No.: 18/922,527

(22) Filed: Oct. 22, 2024

Related U.S. Application Data
(63) Continuation of application No. PCT/JP2023/013660, filed on Mar. 31, 2023.

(30) Foreign Application Priority Data
May 17, 2022 (JP) 2022-081186

(51) Int. Cl. G11B 7/005; C07C 13/48

(52) U.S. Cl. CPC G11B 7/005; C07C 13/48; C07C 2602/10

(57) ABSTRACT

A non-linear optical-absorbing material in an aspect of the present disclosure contains a compound represented by Formula (1) below:

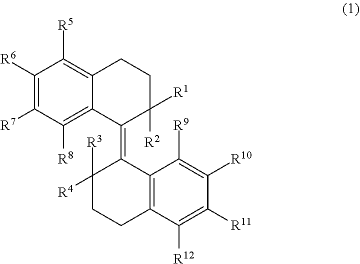

in Formula (1) above, R¹ to R¹² mutually independently represent a group containing at least one atom selected from the group consisting of H, B, C, N, O, F, Si, P, S, Cl, I, and Br.

### BACKGROUND

### 1. Technical Field

The present disclosure relates to a non-linear optical-absorbing material, a recording medium, a method for recording information, and a method for reading information.

### 2. Description of the Related Art

Among optical materials such as optical-absorbing materials, a material having a non-linear optical effect is called a non-linear optical material. The non-linear optical effect means that when strong light such as laser light is applied to a substance, an optical phenomenon proportional to the square or a higher order than the square of the electric field of the applied light occurs in the substance. Examples of the optical phenomenon include absorption, reflection, scattering, and emission. Examples of a second-order non-linear optical effect, which is proportional to the square of the electric field of the applied light, include second harmonic generation (SHG), the Pockels effect, and the parametric effect. Examples of a third-order non-linear optical effect, which is proportional to the cube of the electric field of the applied light, include two-photon absorption, multiphoton absorption, third harmonic generation (THG), and the Kerr effect. In the present specification, multiphoton absorption such as two-photon absorption may be called non-linear optical absorption. A material that can undergo non-linear optical absorption may be called a non-linear optical-absorbing material. In particular, a material that can undergo two-photon absorption may be called a two-photon-absorbing material. Non-linear optical absorption may be called non-linear absorption.

Many studies on the non-linear optical material have been so far actively advanced. In particular, as the non-linear optical material, inorganic materials that can easily prepare single crystals are being developed. In recent years, development of non-linear optical materials containing organic materials has been expected. Organic materials not only have a higher degree of freedom of design but also have larger non-linear optical constants than those of inorganic materials. Further, in organic materials, non-linear response is performed at high speed. In the present specification, a non-linear optical material containing an organic material may be called an organic non-linear optical material.

### SUMMARY

A new compound having a non-linear optical absorption characteristic against light having a wavelength in a short-wavelength range is demanded.

In one general aspect, the techniques disclosed here feature a non-linear optical-absorbing material containing a compound represented by Formula (1) below:

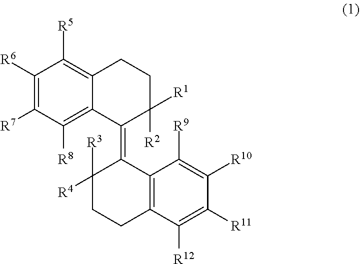

in Formula (1) above, R¹ to R¹² mutually independently represent a group containing at least one atom selected from the group consisting of H, B, C, N, O, F, Si, P, S, Cl, I, and Br.

The present disclosure provides a new compound having a non-linear optical absorption characteristic against light having a wavelength in a short-wavelength range.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a flowchart about a method for recording information using a recording medium containing a non-linear optical-absorbing material according an embodiment of the present disclosure;

FIG. 1B is a flowchart about a method for reading information using the recording medium containing the non-linear optical-absorbing material according to the embodiment of the present disclosure;

FIG. 2 is a graph showing a ¹H-NMR spectrum of a compound represented by Formula (2); and

FIG. 3 is a graph showing a recording and reproduction characteristic of resin thin films of examples and comparative examples.

### DETAILED DESCRIPTIONS

### Underlying Knowledge Forming Basis of the Present Disclosure

In the organic non-linear optical material, the two-photon-absorbing material is particularly attracting attention. Two-photon absorption means a phenomenon in which a compound absorbs two photons almost simultaneously to transition to an excited state. As two-photon absorption, simultaneous two-photon absorption and stepwise two-photon absorption are known. Simultaneous two-photon absorption may be called non-resonant two-photon absorption. Simultaneous two-photon absorption means two-photon absorption in a wavelength range in which there is no absorption band for one photon. Stepwise two-photon absorption may be called resonant two-photon absorption. In stepwise two-photon absorption, the compound absorbs a first photon and then further absorbs a second photon to transition to a higher-order excited state. In stepwise two-photon absorption, the compound absorbs the two photons successively.

In simultaneous two-photon absorption, an optical absorption amount by the compound is usually proportional to the square of applied light intensity to show non-linearity. The optical absorption amount by the compound can be used as an indicator of the efficiency of two-photon absorption. When the optical absorption amount by the compound shows non-linearity, for example, optical absorption by the compound can be caused to occur only near the focal point of laser light having high electric field strength. That is, in a sample containing the two-photon-absorbing material, the compound can be excited only at a desired position. Thus, the compound in which simultaneous two-photon absorption occurs brings about extremely high spatial resolution and is thus considered to be applied for uses such as recording layers of three-dimensional optical memories and photocurable resin compositions for three-dimensional (3D) laser microfabrication. When the two-photon-absorbing material further has a fluorescent characteristic, the two-photon-absorbing material can also be applied for fluorescent dye materials for use in two-photon fluorescent microscopes or the like. When this two-photon-absorbing material is used for three-dimensional optical memories, there is a possibility that a system reading an on/off state of a recording layer based on a change in fluorescence from the two-photon-absorbing material can be employed. Current optical memories employ a system reading the on/off state of the recording layer based on a change in the reflection rate of light and a change in the absorption rate of light in the two-photon-absorbing material. However, when this system is used for three-dimensional optical memories, conventional two-photon-absorbing materials have smaller two-photon absorption efficiency than one-photon absorption efficiency, and thus crosstalk may occur based on another recording layer different from the recording layer from which the on/off state is to be read.

In the two-photon-absorbing material, a two-photon absorption cross section (a GM value) is used as an indicator indicating the efficiency of two-photon absorption. The unit of the two-photon absorption cross section is GM (10⁻⁵⁰ cm⁴·s·molecule⁻¹·photon⁻¹). Many organic two-photon-absorbing materials having a large two-photon absorption cross section have been so far developed. For example, many compounds having a two-photon absorption cross section large enough to be greater than 500 GM have been reported (for example, Harry L. Anderson et al, “Two-Photon Absorption and the Design of Two-Photon Dyes”, Angew. Chem. Int. Ed. 2009, Vol. 48, p. 3244-3266). However, in almost all reports, the two-photon absorption cross section has been measured using laser light having a wavelength longer than 600 nm. In particular, even a near-infrared ray having a wavelength longer than 750 nm is used as the laser light.

However, to apply the two-photon-absorbing material to industrial uses, a material exhibiting a two-photon absorption characteristic when laser light having a shorter wavelength is applied is required. For example, in the field of three-dimensional optical memories, the laser light having a shorter wavelength can achieve a finer concentrated spot and can thus improve the recording density of three-dimensional optical memories. In the field of three-dimensional (3D) laser microfabrication too, the laser light having a shorter wavelength can achieve shaping with higher resolution. Further, in the standard of Blu-ray (registered trademark) Disc, laser light having a center wavelength of 405 nm is used. Thus, if a compound having an excellent two-photon absorption characteristic against light in the same wavelength range as that of the laser light having a shorter wavelength is developed, it can significantly contribute to industrial development.

Further, a light-emitting apparatus emitting ultrashort pulsed laser having high light intensity tends to be large in size and unstable in operation. Thus, it is difficult to employ such a light-emitting apparatus for industrial uses from the viewpoints of versatility and reliability. Considering this, to apply the two-photon-absorbing material to industrial uses, a material exhibiting the two-photon absorption characteristic even when laser light with low light intensity is applied.

As a light source, for example, a femtosecond laser such as a titanium sapphire laser or a pulsed laser having a pulse width ranging from picoseconds to nanoseconds, such as a semiconductor laser, can be used. From the viewpoints of being small in size, highly versatile, and stable in operation, the semiconductor laser is suitable for industrial uses.

When light, using a laser with a pulse width of picoseconds to nanoseconds or greater, concentrated by a lens to have increased photon density, is applied to the organic non-linear optical material, electrons undergo one-photon excitation or two-photon excitation in the order of femtoseconds and relax to the lowest excited state in a few hundred femtoseconds to picoseconds. Even a point in time when the electrons have relaxed to the lowest excited state is in the middle of pulsed application. Thus, excitation from the lowest excited state to a higher-order excited state may occur. This phenomenon is called excited state absorption (ESA). Subsequently, so long as pulsed application continues, excited state absorption and relaxation to the lowest excited state are repeated. This relaxation is a very rapid process that is completed in the order of picoseconds at the slowest and is non-radiative deactivation except special cases such as azulene. That is, not deactivation by emitting light such as fluorescence or phosphorescence, but deactivation occurs by emitting heat. When the non-linear optical-absorbing material is thus locally excited using the laser with a pulse width of picoseconds to nanoseconds or greater, when the non-linear optical-absorbing material further causes excited state absorption, it is possible to locally generate heat. This enables, for example, the non-linear optical-absorbing material to be used as a heat source for locally altering a recording medium and, in turn, enables three-dimensional recording.

Excited state absorption proceeds as follows, for example.

(1-1) Through one-photon absorption or two-photon absorption, electrons transition from the ground state (S₀) to an excited state and quickly relax to the first excited state (singlet, S₁).
(1-2) Through further one-photon absorption from the S₁ state, the electrons are excited to a higher-order singlet excited state (Sₙ).

### Alternatively,

(2-1) Through one-photon absorption or two-photon absorption, electrons transition from the ground state (S₀) to an excited state and quickly relax to the first excited state (singlet, S₁).
(2-2) Intersystem crossing (ISC) from the S₁ state to the triplet excited state (T₁) occurs.
(2-3) Through further one-photon absorption from the T₁ state, the electrons are excited to a higher-order triplet excited state (Tₙ).

When excited state absorption does not occur, for example, electron transition proceeds as follows.

(3-1) Through one-photon absorption or two-photon absorption, electrons transition from the ground state (S₀) to an excited state and quickly relax to the first excited state (singlet, S₁).
(3-2) The electrons transition from the S₁ state to the S₀ state to deactivate.

### Alternatively,

(4-1) Through one-photon absorption or two-photon absorption, electrons transition from the ground state (S₀) to an excited state and quickly relax to the first excited state (singlet, S₁).
(4-2) Intersystem crossing from the S₁ state to the triplet excited state (T₁) occurs.
(4-3) The electrons relax from the T₁ state to the S₀ state to deactivate.

As described above, excited state absorption is successive multiphoton absorption and is a kind of non-linear optical absorption. Excited state absorption also occurs only when high-intensity light is applied to a sample as in two-photon absorption. The probability of the occurrence of excited state absorption when low-intensity light is applied to the sample is small enough to neglect.

In a compound having a non-linear optical absorption characteristic, the relation between light intensity and an absorption characteristic when laser light with a pulse width of picoseconds to nanoseconds or greater is applied is represented by Expressions (i) and (ii) below. In the present specification, the compound having a non-linear optical absorption characteristic may be called a non-linear optical-absorbing compound. Expressions (i) and (ii) are mathematical expressions for calculating a light intensity reduction −dI when light with intensity I is applied to a sample containing the non-linear optical-absorbing compound and having a microscopic thickness dz.

$$- \frac{dI}{dz} = AI + {BI}^{2} + {CI}^{3} \qquad ( i )$$

$$A = α , B = β + α \frac{{σ}_{ESA} τ}{2 ℏω} , C = γ + β \frac{{σ}_{ESA} τ}{2 ℏω} \qquad ( ii )$$

In Expression (ii), α is a one-photon absorption coefficient (cm⁻¹); β is a simultaneous two-photon absorption coefficient (cm/W); γ is a simultaneous three-photon absorption coefficient (cm³/W²); σ_{ESA} is an excited state absorption cross section (cm²); τ is the excited-state lifetime (s); h−(h bar) is the Dirac constant (J·s); and ω is the angular frequency (rad/s) of incident light.

Further, α and β can be represented by Expressions (iii) and (iv) below, respectively. In Expressions (iii) and (iv), F is a molar extinction coefficient (mol⁻¹·L·cm⁻¹); N is the number of molecules (mol·cm⁻³) of the compound of the sample per unit volume; N_{A} is Avogadro's constant; and σ is a two-photon absorption cross section (GM).

$$α = 1000 \cdot ln 10 \cdot ε \cdot \frac{N}{{N}_{A}} \qquad ( iii )$$

$$β = \frac{σ N}{ℏω} \qquad ( iv )$$

The absorption coefficient (cm⁻¹) refers to the ratio of photons absorbed per unit length when light travels in a substance. The molar extinction coefficient (mol⁻¹·L·cm⁻¹) refers to the ratio of photons absorbed by 1 mol of molecules when light travels in a substance. The absorption cross section (cm²) refers to the ratio of photons absorbed by one particle (molecule) when light travels in a substance. The absorption coefficient can be converted into the absorption cross section by being divided by the number of molecules of the sample per unit volume (the number density of molecules). The absorption cross section can be converted into the molar extinction coefficient by multiplying it by Avogadro's constant (6.02×10²³ mol⁻¹) to perform unit conversion.

When applied light intensity is low, the contribution of third-order non-linear optical absorption is small. For example, when the light of a small-sized, highly versatile semiconductor laser is applied, the third-order term in Expression (i) is considered to be small enough to neglect. For simplicity, the following describes the non-linearity of optical absorption using Expression (v) below, which neglects CI³.

$$- \frac{dI}{dz} = AI + {BI}^{2} \qquad ( v )$$

It can be seen from Expression (v) that in the sample, the intensity I of incident light when a linear absorption amount (the first-order term: AI) and a non-linear absorption amount (the second-order term: BI²) are equal to each other is represented by A/B. That is, when the intensity I of the incident light is smaller than A/B, in the sample, linear optical absorption preferentially occurs. When the intensity I of the incident light is larger than A/B, in the sample, non-linear optical absorption preferentially occurs. Thus, there is a tendency that the smaller the value of A/B in the sample, the more non-linear optical absorption can be preferentially exhibited by laser light with low light intensity. Here, A/B is represented by Expression (vi) below:

$$A / B = α / ( β + α \frac{{σ}_{ESA} τ}{2 ω} ) \qquad ( vi )$$

In the case of a material in which excited state absorption does not occur, σ_{ESA}0, and thus Expression (vii) below holds. Thus, when the incident light intensity is smaller than α/β, linear optical absorption occurs preferentially over non-linear optical absorption.

$$α ( \frac{{σ}_{ESA} τ}{2 ω} ) = 0 \qquad ( vii )$$

On the other hand, in the case of a material in which excited state absorption occurs, the term on the left side of Expression (vii) has a value, and Expression (vii) does not hold. Thus, a threshold of the incident light intensity I for causing non-linear optical absorption preferentially over liner optical absorption can be lowered. A material having large excited state absorption can preferentially cause non-linear optical absorption even with extremely low incident light intensity.

Japanese Patent No. 6448042 discloses formation of a recording mark by applying laser with a center wavelength of 401 nm and a pulse width of 8 nanoseconds to an optical information recording material containing a non-linear absorption dye and formed with a multilayer diffraction grating and locally destroying the diffraction grating. As the non-linear absorption dye, disclosed are 1,1,4,4-tetraphenyl-1,3-butadiene, 1,3,6,8-tetraphenylpyrene, pyrene-ethylene glycol-pyrene, 1,4-bis(phenylethyl)benzene, 1,2,4,5-tetrakis(phenylethyl)benzene, 9,10-diphenylanthracene, 5,6,11,12-tetraphenylnaphthacene, fluorene, 2,7-dibromofluorene, 1-bromopyrene, 4-bromopyrene, and pyrene.

Japanese Patent No. 5738554 discloses a hologram recording medium containing a non-linear sensitizer transitioning to a higher-order triplet exited state by laser application with a wavelength of 405 nm and a pulse width of 5 nanoseconds. As the non-linear sensitizer, a platinum-ethynyl complex is disclosed.

However, if the absorption coefficient of the non-linear absorption dye in the ground state is too small, the value of −dI/dz in Expression (v) is small, making recording sensitivity insufficient. When the non-linear absorption dye absorbs light to be excited and then passes through the triplet excited state before returning to the ground state, there is a concern that lightfastness is insufficient. This is because oxygen molecules in the atmosphere are present as a triplet state in the ground state to cause an energy transfer reaction with the dye in the triplet excited state to generate singlet oxygen. The triplet excited state involves spin inversion when returning to the ground state and thus has long life; it has an excited-state lifetime in the order of a few hundred milliseconds if it is extremely long. The larger the number of dye molecules passing through the triplet excited state after being excited, that is, the higher the quantum yield of the intersystem crossing of the dye, the higher the probability of reacting with the oxygen molecules. The longer the triplet excited-state lifetime, the even higher the probability of reacting with the oxygen molecules. Singlet oxygen is electron-deficient, has extremely high activity, and reacts with the dye or a polymer compound present therearound to alter it. The dye that has reacted with singlet oxygen undergoes changes in optical characteristics such as discoloration.
The present inventors have conducted earnest study to newly find that a compound represented by Formula (1) described below has a high non-linear optical absorption characteristic against light having a wavelength in a short-wavelength range. Specifically, the present inventors have found that in the compound represented by Formula (1), the one-photon absorption coefficient α is in a range of being not too large and not too small against the light having a wavelength in a short-wavelength range, and further the excited state absorption cross section σ_{ESA} has a large value. Consequently, in the compound represented by Formula (1), the value of −dI/dz described above is sufficiently large, and in addition, the value of the ratio A/B of the nonlinear optical absorption coefficient B in the linear term of equation (vi) to the linear optical absorption coefficient A in the quadratic term of equation (vi) is small, and the compound tends to have high non-linearly in optical absorption. In the compound represented by Formula (1), excited state absorption occurring in the short-wavelength range is one from the singlet excited state, and the excited-state lifetime is not too long, making it hard to cause a reaction with oxygen in the atmosphere. Thus, alteration by the generation of singlet oxygen is also hard to occur. In the present specification, the short-wavelength range means a wavelength range including 405 nm and means, for example, a wavelength range of longer than or equal to 390 nm and shorter than or equal to 420 nm.

### SUMMARY OF ASPECT ACCORDING TO PRESENT DISCLOSURE

A non-linear optical-absorbing material according to a first aspect of the present disclosure contains a compound represented by Formula (1) below:

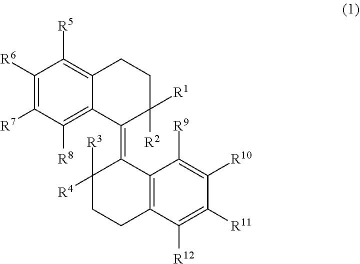

in Formula (1) above, R¹ to R¹² mutually independently represent a group containing at least one atom selected from the group consisting of H, B, C, N, O, F, Si, P, S, Cl, I, and Br.

The non-linear optical-absorbing material according to the first aspect has a non-linear optical absorption characteristic against light having a wavelength in a short-wavelength range.

In a second aspect of the present disclosure, for example, in the non-linear optical-absorbing material according to the first aspect, R⁵ to R¹² may each be a hydrogen atom.

In a third aspect of the present disclosure, for example, in the non-linear optical-absorbing material according to the first or second aspect, R¹ and R² may be the same group or R¹ and R³ may be the same group.

In a fourth aspect of the present disclosure, for example, in the non-linear optical-absorbing material according to any one of the first to third aspects, R¹ to R⁴ may be the same group.

According to the second to fourth aspects, synthesis of the compound represented by Formula (1) is easy.

In a fifth aspect of the present disclosure, for example, in the non-linear optical-absorbing material according to any one of the first to fourth aspects, R¹ to R⁴ may each be a methyl group.

According to the fifth aspect, the compound represented by Formula (1) can stably exhibit its original characteristic.

In a sixth aspect of the present disclosure, for example, the non-linear optical-absorbing material according to any one of the first to fifth aspects may be used for a device using light having a wavelength of longer than or equal to 390 nm and shorter than or equal to 420 nm.

According to the sixth aspect, the non-linear optical-absorbing material is suitable for the use of the device using the light having a wavelength of longer than or equal to 390 nm and shorter than or equal to 420 nm.

A recording medium according to a seventh aspect of the present disclosure contains the non-linear optical-absorbing material according to any one of the first to sixth aspects.

According to the seventh aspect, in the non-linear optical-absorbing material, the non-linear optical absorption characteristic against the light having a wavelength in a short-wavelength range is improved. The recording medium containing such a non-linear optical-absorbing material can record information with high recording density.

A method for recording information according to an eighth aspect of the present disclosure includes:
preparing a light source emitting light having a wavelength of longer than or equal to 390 nm and shorter than or equal to 420 nm; and
concentrating the light from the light source and applying the light to a recording layer of a recording medium containing the non-linear optical-absorbing material according to any one of the first to seventh aspects.

According to the eighth aspect, in the non-linear optical-absorbing material, the non-linear optical absorption characteristic against the light having a wavelength in a short-wavelength range is improved. According to the method for recording information using the recording medium containing such a non-linear optical-absorbing material, information can be recorded with high recording density.

A method for reading information according to a ninth aspect of the present disclosure is, for example, a method for reading information recorded by the method for recording information according to the eighth aspect, the method for reading information including:
applying light to the recording layer of the recording medium to measure an optical characteristic of the recording layer; and
reading information from the recording layer.

In a 10th aspect of the present disclosure, for example, in the method for reading information according to the ninth aspect, the optical characteristic may be intensity of light reflected by the recording layer.

According to the ninth or 10th aspect, when information is read, the occurrence of crosstalk based on another recording layer can be prevented.

The following describes an embodiment of the present disclosure with reference to the accompanying drawings. The present disclosure is not limited to the following embodiment.

### EMBODIMENT

A non-linear optical-absorbing material according to the present embodiment contains a compound a represented by Formula (1) below:

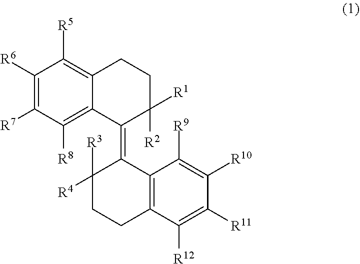

In Formula (1), R¹ to R¹² mutually independently represent a group containing at least one atom selected from the group consisting of H, B, C, N, O, F, Si, P, S, Cl, I, and Br.

The compound a has a sufficiently large absorption amount of the light having a wavelength in a short-wavelength range. As the breakdown of the absorption amount, there is a tendency that the value of the ratio A/B of the magnitude A of liner optical absorption to the magnitude B of non-linear optical absorption is small, that is, a tendency that the non-linearity of optical absorption is high. The compound a does not pass through the triplet excited state in its relaxation process and thus has excellent lightfastness. Thus, the compound a is improved in view of achieving both the non-linear optical absorption characteristic against the light having a wavelength in a short-wavelength range and lightfastness. Further, recording sensitivity is improved. The compound a is excited by application of laser light having a wavelength in a short-wavelength range, relaxes to the lowest singlet excited state with structural change, and further absorbs laser light from the lowest singlet excited state to transition to a higher-order singlet excited state. The structural change after excitation is caused by a double bond connecting two six-member rings to each other twisting. In an adiabatic state, the π-electron conjugated system becomes short. This makes the optical absorption band of the excited state blue-shift to a short-wavelength range, and σ_{ESA} in the short-wavelength range shows a large value.

In Formula (1), R¹ to R¹² may mutually independently be a hydrogen atom, a halogen atom, a hydrocarbon group, a halogenated hydrocarbon group, a group containing an oxygen atom, a group containing a nitrogen atom, a group containing a sulfur atom, a group containing a silicon atom, a group containing a phosphorus atom, or a group containing a boron atom,

Examples of the halogen atom include F, Cl, Br, and I. In the present specification, the halogen atom may be called a halogen group.

The hydrocarbon group is an alkyl group or an unsaturated hydrocarbon group.

The number of carbon atoms in the alkyl group, which is not particularly limited, is, for example, greater than or equal to one and less than or equal to 20. The number of carbon atoms in the alkyl group may be greater than or equal to one and less than or equal to 10 or greater than or equal to one and less than or equal to five from the viewpoint of capability of easily synthesizing the compound a. By adjusting the number of carbon atoms in the alkyl group, solubility against a solvent or a resin composition can be adjusted for the compound a. The alkyl group may be linear, branched, or cyclic. At least one hydrogen atom contained in the alkyl group may be replaced by a group containing at least one atom selected from the group consisting of N, O, P, and S. Examples of the alkyl group include a methyl group, an ethyl group, a propyl group, a butyl group, a 2-methylbutyl group, a pentyl group, a hexyl group, a 2,3-dimethylhexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an eicosyl group, a 2-methoxybutyl group, and a 6-methoxyhexyl group.

The unsaturated hydrocarbon group contains unsaturated bonds such as a carbon-carbon double bond and a carbon-carbon triple bond. The number of the unsaturated bonds contained in the unsaturated hydrocarbon group is, for example, greater than or equal to one and less than or equal to five. The number of carbon atoms in the unsaturated hydrocarbon group, which is not particularly limited, may be, for example, greater than or equal to two and less than or equal to 20, greater than or equal to two and less than or equal to 10, or greater than or equal to two and less than or equal to five. The unsaturated hydrocarbon group may be linear, branched, or cyclic. At least one hydrogen atom contained in the unsaturated hydrocarbon group may be replaced by a group containing at least one atom selected from the group consisting of N, O, P, and S. Examples of the unsaturated hydrocarbon group include a vinyl group, an ethynyl group, and an allyl group.

The halogenated hydrocarbon group means a group in which at least one hydrogen atom contained in a hydrocarbon group is replaced by a halogen atom. The halogenated hydrocarbon group may be a group in which all the hydrogen atoms contained in the hydrocarbon group may be replaced by halogen atoms. Examples of the halogenated hydrocarbon group include a halogenated alkyl group and a halogenated alkenyl group.

Examples of the halogenated alkyl group include —CF₃, —CH₂F, —CH₂Br, —CH₂Cl, —CH₂I, and —CH₂CF₃. Examples of the halogenated alkenyl group include —CH═CHCF₃.

The group containing an oxygen atom is, for example, a substituent having at least one selected from the group consisting of a hydroxy group, a carboxy group, an aldehyde group, an ether group, an acyl group, and an ester group.

Examples of the substituent having a hydroxy group include a hydroxy group itself and a hydrocarbon group having a hydroxy group. In this substituent, the hydroxy group may be deprotonated to be a state of —O⁻. Examples of the hydrocarbon group having a hydroxy group include —CH₂OH, —CH(OH)CH₃, —CH₂CH(OH)CH₃, and —CH₂C(OH)(CH₃)₂.

Examples of the substituent having a carboxy group include a carboxy group itself and a hydrocarbon group having a carboxy group. In this substituent, the carboxy group may be deprotonated to be a state of —CO₂⁻. Examples of the hydrocarbon group having a carboxy group include —CH₂CH₂COOH, —C(COOH)(CH₃)₂, and —CH₂CO₂⁻.

Examples of the substituent having an aldehyde group include an aldehyde group itself and a hydrocarbon group having an aldehyde group. Examples of the hydrocarbon group having an aldehyde group include —CH═CHCHO.

Examples of the substituent having an ether group include an alkoxy group, a halogenated alkoxy group, an alkenyloxy group, an oxiranyl group, and a hydrocarbon group having at least one of these functional groups. At least one hydrogen atom contained in the alkoxy group may be replaced by a group containing at least one atom selected from the group consisting of N, O, P, and S. Examples of the alkoxy group include a methoxy group, an ethoxy group, a 2-methoxyethoxy group, a butoxy group, a 2-methylbutoxy group, a 2-methoxybutoxy group, a 4-ethylthiobutoxy group, a pentyloxy group, a hexyloxy group, a heptyloxy group, an octyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyloxy group, an eicosyloxy group, —OCH₂O⁻, —OCH₂CH₂O⁻, and —O(CH₂)₃O⁻. Examples of the halogenated alkoxy group include —OCHF₂, —OCH₂F, and —OCH₂Cl. Examples of the alkenyloxy group include —OCH═CH₂.

Examples of the hydrocarbon group having a functional group such as an alkoxy group include —CH₂OCH₃, —C(OCH₃)₃, a 2-methoxybutyl group, and a 6-methoxyhexyl group.

Examples of the substituent having an acyl group include an acyl group itself and a hydrocarbon group having an acyl group. Examples of the acyl group include —COCH₃. Examples of the hydrocarbon group having an acyl group include —CH═CHCOCH₃.

Examples of the substituent having an ester group include an alkoxycarbonyl group, an acyloxy group, and a hydrocarbon group having at least one of these functional groups. Examples of the alkoxycarbonyl group include —COOCH₃, —COO(CH₂)₃CH₃, and —COO(CH₂)₇CH₃. Examples of the acyloxy group include —OCOCH₃. Examples of the hydrocarbon group having a functional group such as an acyloxy group include —CH₂OCOCH₃.

The group containing a nitrogen atom is, for example, a substituent having at least one selected from the group consisting of an amino group, an imino group, a cyano group, an azi group, an amide group, a carbamate group, a nitro group, a cyanamide group, an isocyanate group, and an oxime group.

Examples of the substituent having an amino group include a primary amino group, a secondary amino group, a tertiary amino group, a quarternary amino group, and a hydrocarbon group having at least one of these functional groups. In this substituent, the amino group may be protonated. Examples of the tertiary amino group include —N(CH₃)₂.

Examples of the hydrocarbon group having a functional group such as a primary amino group include —CH₂NH₂, —CH₂N(CH₃)₂, —(CH₂)₄N(CH₃)₂, —CH₂CH₂NH₃⁺, —CH₂CH₂NH(CH₃)₂⁺, and —CH₂CH₂N(CH₃)₃⁺.

Examples of the substituent having an imino group include an imino group itself and a hydrocarbon group having an imino group. Examples of the imino group include —N═CCl₂.

Examples of the substituent having a cyano group include a cyano group itself and a hydrocarbon group having a cyano group. Examples of the hydrocarbon group having a cyano group include —CH₂CN and —CH═CHCN.

Examples of the substituent having an azi group include an azi group itself and a hydrocarbon group having an azi group.

Examples of the substituent having an amide group include an amide group itself and a hydrocarbon group having an amide group. Examples of the amide group include —CONH₂, —NHCHO, —NHCOCH₃, —NHCOCF₃, —NHCOCH₂Cl, and —NHCOCH(CH₃)₂. Examples of the hydrocarbon group having an amide group include —CH₂CONH₂ and —CH₂NHCOCH₃.

Examples of the substituent having a carbamate group include a carbamate group itself and a hydrocarbon group having a carbamate group. Examples of the carbamate group include —NHCOOCH₃, —NHCOOCH₂CH₃, and —NHCO₂(CH₂)₃CH₃.

Examples of the substituent having a nitro group include a nitro group itself and a hydrocarbon group having a nitro group. Examples of the hydrocarbon group having a nitro group include —C(NO₂)(CH₃)₂.

Examples of the substituent having a cyanamide group include a cyanamide group itself and a hydrocarbon group having a cyanamide group. The cyanamide group is represented by —NHCN.

Examples of the substituent having an isocyanate group include an isocyanate group itself and a hydrocarbon group having an isocyanate group. The isocyanate group is represented by —N═C═O.

Examples of the substituent having an oxime group include an oxime group itself and a hydrocarbon group having an oxime group. The oxime group is represented by —CH═NOH.

The group containing a sulfur atom is, for example, a substituent having at least one selected from the group consisting of a thiol group, a sulfide group, a sulfinyl group, a sulfonyl group, a sulfino group, a sulfonic acid group, an acylthio group, a sulfenamide group, a sulfonamide group, a thioamide group, a thiocarbamide group, and a thiocyano group.

Examples of the substituent having a thiol group include a thiol group itself and a hydrocarbon group having a thiol group. The thiol group is represented by —SH.

Examples of the substituent having a sulfide group include an alkylthio group, an alkyldithio group, an alkenylthio group, an alkynylthio group, a thiacyclopropyl group, and a hydrocarbon group having at least one of these functional groups. At least one hydrogen atom contained in the alkylthio group may be replaced by a halogen group. Examples of the alkylthio group include —SCH₃, —S(CH₂)F, —SCH(CH₃)₂, and —SCH₂CH₃. Examples of the alkyldithio group include —SSCH₃. Examples of the alkenylthio group include —SCH═CH₂ and —SCH₂CH═CH₂. Examples of the alkynylthio group include —SC≡CH.

Examples of the hydrocarbon group having a functional group such as an alkylthio group include —CH₂SCF₃.

Examples of the substituent having a sulfinyl group include a sulfinyl group itself and a hydrocarbon group having a sulfinyl group. Examples of the sulfinyl group include —SOCH₃.

Examples of the substituent having a sulfonyl group include a sulfonyl group itself and a hydrocarbon group having a sulfonyl group. Examples of the sulfonyl group include —SO₂CH₃. Examples of the hydrocarbon group having a sulfonyl group include —CH₂SO₂CH₃ and —CH₂SO₂CH₂CH₃.

Examples of the substituent having a sulfino group include a sulfino group itself and a hydrocarbon group having a sulfino group. In this substituent, the sulfino group may be deprotonated to be a state of —SO₂⁻.

Examples of the substituent having a sulfonic acid group include a sulfonic acid group itself and a hydrocarbon group having a sulfonic acid group. In this substituent, the sulfonic acid group may be deprotonated to be a state of —SO₃⁻.

Examples of the substituent having an acylthio group include an acylthio group itself and a hydrocarbon group having an acylthio group. Examples of the acylthio group include —SCOCH₃.

Examples of the substituent having a sulfenamide group include a sulfenamide group itself and a hydrocarbon group having a sulfenamide group. Examples of the sulfenamide group include —SN(CH₃)₂.

Examples of the substituent having a sulfonamide group include a sulfonamide group itself and a hydrocarbon group having a sulfonamide group. Examples of the sulfonamide group include —SO₂NH₂ and —NHSO₂CH₃.

Examples of the substituent having a thioamide group include a thioamide group itself and a hydrocarbon group having a thioamide group. Examples of the thioamide group include —NHCSCH₃. Examples of the hydrocarbon group having a thioamide group include —CH₂SC(NH₂)₂⁺.

Examples of the substituent having a thiocarbamide group include a thiocarbamide group itself and a hydrocarbon group having a thiocarbamide group. Examples of the thiocarbamide group include —NHCSNHCH₂CH₃.

Examples of the substituent having a thiocyano group include a thiocyano group itself and a hydrocarbon group having a thiocyano group. Examples of the hydrocarbon group having a thiocyano group include —CH₂SCN.

The group containing a silicon atom is, for example, a substituent having at least one selected from the group consisting of a silyl group and a siloxy group.

Examples of the substituent having a silyl group include a silyl group itself and a hydrocarbon group having a silyl group. Examples of the silyl group include —Si(CH₃)₃, —SiH(CH₃)₂, —Si(OCH₃)₃, —Si(OCH₂CH₃)₃, —SiCH₃(OCH₃)₂, —Si(CH₃)₂OCH₃, —Si(N(CH₃)₂)₃, —SiF(CH₃)₂, —Si(OSi(CH₃)₃)₃, and —Si(CH₃)₂OSi(CH₃)₃. Examples of the hydrocarbon group having a silyl group include —(CH₂)₂Si(CH₃)₃.

Examples of the substituent having a siloxy group include a siloxy group itself and a hydrocarbon group having a siloxy group. Examples of the hydrocarbon group having a siloxy group include —CH₂OSi(CH₃)₃.

The group containing a phosphorus atom is, for example, a substituent having at least one selected from the group consisting of a phosphino group and a phosphoryl group.

Examples of the substituent having a phosphino group include a phosphino group itself and a hydrocarbon group having a phosphino group. Examples of the phosphino group include —PH₂, —P(CH₃)₂, —P(CH₂CH₃)₂, —P(C(CH₃)₃)₂, and —P(CH(CH₃)₂)₂.

Examples of the substituent having a phosphoryl group include a phosphoryl group itself and a hydrocarbon group having a phosphoryl group. Examples of the hydrocarbon group having a phosphoryl group include —CH₂PO(OCH₂CH₃)₂.

The group containing a boron atom is, for example, a substituent having a boronic acid group. Examples of the substituent having a boronic acid group include a boronic acid group itself and a hydrocarbon group having a boronic acid group.

In Formula (1), R⁵ to R¹² may each be a hydrogen atom. In this case, the aromatic ring of the compound a represented by Formula (1) does not have any substituent. This can prevent rising of the energy of the highest occupied molecular orbital (HOMO) of the compound and reducing of the energy of the lowest unoccupied molecular orbital (LUMO) caused by the electron-withdrawing property or the electron-donating property of the substituent. That is, the energy gap between HOMO and LUMO can be prevented from reducing. This can prevent a peak derived from one-photon absorption from shifting to a longer wavelength and can prevent the value of ratio A/B of the nonlinear optical absorption coefficient B in the linear term of equation (vi) to the linear optical absorption coefficient A in the quadratic term of equation (vi) from increasing.

In Formula (1), R¹ and R² may be the same group. Alternatively, R¹ and R³ may be the same group. This configuration makes synthesis of the compound represented by Formula (1) easy.

In Formula (1), R¹ to R⁴ may be the same group. This configuration makes synthesis of the compound represented by Formula (1) easy.

In Formula (1), R¹ to R⁴ may each be a hydrocarbon group or a halogenated hydrocarbon group having less than or equal to five carbon atoms. R¹ to R⁴ may each be a methyl group or a CF₃ group.

In Formula (1), R¹ to R¹² may each be a group that does not contain any aromatic ring.

Specifically, the compound contained in the non-linear optical-absorbing material may be represented by Formula (2) below:

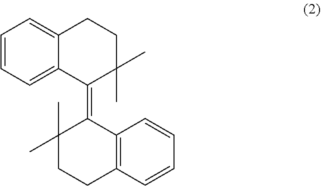 [SMILES: CC1(C)CCc2ccccc2/C1=C1/c2ccccc2CCC1(C)C]

The compound represented by Formula (2) has a cis form and a trans form as isomers. Compared to a compound having hydrogen atoms for all R¹ to R⁴ in Formula (1), the cis form is less stable due to steric hindrance. Even when being isomerized by light application, the compound represented by Formula (2) quickly returns to the trans form at room temperature. Due to this characteristic, a trans:cis ratio obtained at the time of synthesis is 100:0 (Michael Oelgemoller et al, “Synthesis, structural characterization and photoisomerization of cyclic stilbenes”, Tetrahedron, 2012, 68, 4048-4056). Thus, materials or devices containing the compound represented by Formula (2) are not necessarily required to be stored in a dark environment and can stably exhibit their original characteristic.

The method for synthesizing the compound a is not particularly limited, and, for example, the McMurry coupling reaction or the like can be used. The compound a represented by Formula (1) can be synthesized by, for example, the following method. First, a compound b represented by Formula (3) below and a compound c represented by Formula (4) below are prepared.

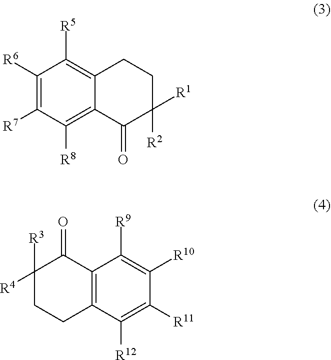

Next, a coupling reaction of the compound b and the compound c is performed. This can synthesize the compound a. The conditions of the coupling reaction can be adjusted appropriately in accordance with, for example, the type of the substituents contained in each of the compound b and the compound c or the like.

The compound b represented by Formula (3) can be synthesized by, for example, the following method. First, a compound d as a tetralone derivative represented by Formula (5) below and halides represented by R¹—X and R²—X are prepared. X is a halogen atom. Examples of the halogen atom include Br and I.

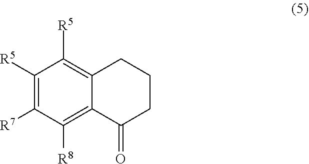

Next, a coupling reaction of the compound d and R¹—X is performed. This can synthesize a compound e represented by Formula (6) below. The conditions of the coupling reaction can be adjusted appropriately in accordance with, for example, the type of the substituents contained in each of the compound d and R¹—X or the like.

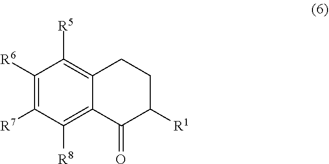

Next, a coupling reaction of the compound e and R²—X is performed. This can synthesize the compound b represented by Formula (3). The conditions of the coupling reaction can be adjusted appropriately in accordance with, for example, the type of the substituents contained in each of the compound e and R²—X or the like.

The compound a represented by Formula (1) has an excellent non-linear optical absorption characteristic against the light having a wavelength in a short-wavelength range. A second-order non-linear absorption coefficient is represented by the sum of the product of the one-photon absorption coefficient, the excited state absorption cross section, and the life of the excited state and the two-photon absorption coefficient.

The two-photon absorption cross section of the compound a against light having a wavelength of 405 nm may be greater than 1 GM, greater than or equal to 10 GM, greater than or equal to 20 GM, greater than or equal to 100 GM, greater than or equal to 400 GM, or greater than or equal to 600 GM. The upper limit of the two-photon absorption cross section of the compound a, which is not particularly limited, is, for example, 10,000 GM and may be 1,000 GM. The two-photon absorption cross section can be measured by, for example, Z scan method described in J. Opt. Soc. Am. B, 2003, Vol. 20, p. 529. Z scan method is widely being used as a method for measuring non-linear optical constants. In Z scan method, near a focal point at which a laser beam is concentrated, a sample to be measured is moved along the application direction of the beam. In this process, a change in the light amount of the light having passed through the sample to be measured is recorded. In Z scan method, the power density of incident light changes in accordance with the position of the sample to be measured. Thus, when the sample to be measured undergoes non-linear optical absorption, when the sample to be measured is positioned near the focal point of the laser beam, the light amount of the transmitted light attenuates. By performing fitting for the change in the transmitted light amount against a theoretical curve predicted from the intensity of the incident light, the thickness of the sample to be measured, the concentration of the compound a in the sample to be measured, and the like, the two-photon absorption cross section can be calculated.

The molar extinction coefficient of the compound a against the light having a wavelength of 405 nm is, for example, less than 4,000 mol⁻¹·L·cm⁻¹ and may be less than or equal to 2,000 mol⁻¹·L·cm⁻¹, less than or equal to 1,000 mol⁻¹·L·cm⁻¹, or less than or equal to 500 mol⁻¹·L·cm⁻¹. The lower limit value of the molar extinction coefficient of the compound a, which is not particularly limited, is, for example, 90 mol⁻¹·L·cm⁻¹. The molar extinction coefficient can be measured by, for example, a method conforming to the stipulation of Japanese Industrial Standards (JIS) K0115: 2004. In the measurement of the molar extinction coefficient, a light source emitting light with photon density causing almost no two-photon optical absorption by the compound a is used. Further, in the measurement of the molar extinction coefficient, for example, the concentration of the compound a is adjusted to 1 mmol/L. The molar extinction coefficient can be used as an indicator of one-photon absorption.

When the compound a undergoes two-photon absorption, the compound a absorbs energy about double that of the light applied to the compound a. The wavelength of light having energy about double that of the light having a wavelength of 405 nm is, for example, 200 nm. When light having a wavelength of around 200 nm is applied to the compound a, one-photon absorption may occur in the compound a. Further, in the compound a, one-photon absorption may occur for light having a wavelength near the wavelength range in which two-photon absorption occurs.

The compound a represented by Formula (1) can be used, for example, as a component of an optical-absorbing material. That is, the present disclosure provides, from its another aspect, an optical-absorbing material containing the compound a represented by Formula (1). The optical-absorbing material contains, for example, the compound a as a main component. The “main component” means a component contained most in the optical-absorbing material in terms of weight ratio. The optical-absorbing material, for example, substantially contains the compound a. “Substantially contains” means that other components changing the essential feature of the material referred to are excluded. However, the optical-absorbing material may contain impurities other than the compound a.

The compound a is used for, for example, devices using the light having a wavelength in a short-wavelength range. As an example, the compound a is used for devices using light having a wavelength of longer than or equal to 390 nm and shorter than or equal to 420 nm. Examples of such devices include recording media, shaping machines, and fluorescent microscopes. Examples of the recording media include three-dimensional optical memories. A specific example of the three-dimensional optical memories is a three-dimensional optical disc. Examples of the shaping machines include three-dimensional (3D) laser microfabrication machines such as 3D printers. Examples of the fluorescent microscopes include two-photon fluorescent microscopes. The light used in these devices has high photon density, for example, near their focal point. Power density near the focal point of the light used by the devices is, for example, greater than or equal to 0.1 W/cm² and less than or equal to 1.0×10²⁰ W/cm². The power density near the focal point of this light may be greater than or equal to 1.0 W/cm², greater than or equal to 1.0×10² W/cm², or greater than or equal to 1.0×10¹ W/cm². As a light source of the devices, for example, a femtosecond laser such as a titanium sapphire laser or a pulsed laser having a pulse width of picoseconds to nanoseconds, such as a semiconductor laser, can be used.

The recording medium includes, for example, a thin film called a recording layer. In the recording medium, information is recorded in the recording layer. As an example, the thin film as the recording layer contains the compound a. That is, the present disclosure provides, from its another aspect, a recording medium containing the compound a.

The recording layer may further contain a polymer compound functioning as a binder other than the compound a. The recording medium may include a dielectric layer apart from the recording layer. The recording medium includes, for example, a plurality of recording layers and a plurality of dielectric layers. In the recording medium, the recording layers and the dielectric layers may be alternately laminated on each other.

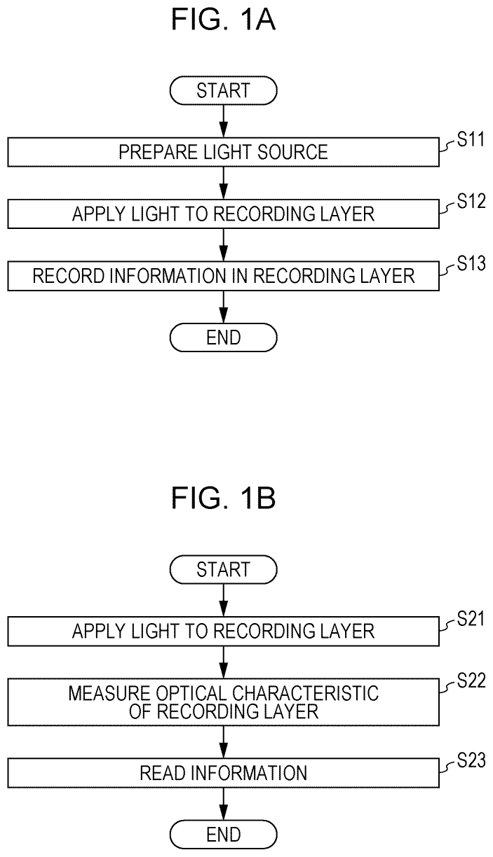

The following describes a method for recording information using the recording medium. FIG. 1A is a flowchart about the method for recording information using the recording medium. First, in Step S11, a light source emitting light having a wavelength of longer than or equal to 390 nm and shorter than or equal to 420 nm is prepared. As the light source, for example, a femtosecond laser such as a titanium sapphire laser or a pulsed laser having a pulse width of picosecond to nanosecond, such as a semiconductor laser, can be used. Next, in Step S12, the light from the light source is concentrated by a lens or the like and is applied to the recording layer of the recording medium. Specifically, the light from the light source is concentrated by a lens or the like and is applied to a recording area of the recording medium. The numerical aperture (NA) of the lens for use in light concentration is not particularly limited. As an example, a lens with an NA in a range of greater than or equal to 0.8 and less than or equal to 0.9 may be used. The power density of this light near its focal point is, for example, greater than or equal to 0.1 W/cm² and less than or equal to 1.0×10²⁰ W/cm². The power density of this light near its focal point may be greater than or equal to 1.0 W/cm², greater than or equal to 1.0×10² W/cm², or greater than or equal to 1.0×10⁵ W/cm². In the present specification, the recording area means a spot that is present in the recording layer and in which information can be recorded by application of light.

In the recording area to which the light has been applied, a physical change or a chemical change occurs. For example, heat occurs when the compound a that has absorbed the light returns from a transition state to the ground state. This heat alters the binder present in the recording area. This changes an optical characteristic of the recording area. For example, the intensity of the light reflected by the recording area, the reflection rate of the light at the recording area, the absorption rate of the light at the recording area, the refractive index of the light at the recording area, or the like changes. The recording area to which the light has been applied may change in the light intensity of fluorescence or the light wavelength of fluorescence emitted from the recording area. This can record information in the recording layer, or specifically, the recording area (Step S13).

The following describes a method for reading information using the recording medium. FIG. 1B is a flowchart about the method for reading information using the recording medium. First, in Step S21, light is applied to the recording layer of the recording medium. Specifically, light is applied to the recording area of the recording medium. The light used in Step S21 may be the same light as the light used for recording the information in the recording medium or different therefrom. Next, in Step S22, an optical characteristic of the recording layer is measured. Specifically, an optical characteristic of the recording area is measured. In Step S22, for example, as the optical characteristic of the recording area, the intensity of the light reflected by the recording area is measured. In Step S22, as the optical characteristic of the recording area, the reflection rate of the light at the recording area, the absorption rate of the light at the recording area, the refractive index of the light at the recording area, the light intensity of fluorescence or the light wavelength of fluorescence emitted from the recording area, or the like may be measured. Next, in Step S23, the information is read from the recording layer, or specifically, the recording area.

In the method for reading information, the recording area in which the information has been recorded can be searched for by the following method. First, light is applied to a specific area of the recording medium. This light may be the same light as the light used for recording the information in the recording medium or different therefrom. Next, an optical characteristic of the area to which the light has been applied is measured. Examples of the optical characteristic include the intensity of the light reflected by the area, the reflection rate of the light at the area, the absorption rate of the light at the area, the refractive index of the light at the area, the light intensity of fluorescence emitted from the area, and the light wavelength of fluorescence emitted from the area. Based on the measured optical characteristic, whether the area to which the light has been applied is the recording area is determined. For example, it is determined that the area is the recording area when the intensity of the light reflected by the area is less than or equal to a specific value. On the other hand, it is determined that the area is not the recording area when the intensity of the light reflected by the area is greater than the specific value. The method for determining whether the area to which the light has been applied is the recording area is not limited to the above method. For example, it may be determined that the area is the recording area when the intensity of the light reflected by the area is greater than a specific value. It may be determined that the area is not the recording area when the intensity of the light reflected by the area is less than or equal to the specific value. When it is determined that the area is not the recording area, the same operation is performed for another area of the recording medium. This can search for the recording area.

The method for recording information and the method for reading information using the recording medium can be performed by, for example, a known recording apparatus. The recording apparatus includes, for example, a light source applying light to the recording area of the recording medium, a measuring device measuring the optical characteristic of the recording area, and a controller controlling the light source and the measuring device.

The shaping machine performs shaping by, for example, applying light to a photocurable resin composition and curing the resin composition. As an example, the photocurable resin composition for three-dimensional (3D) laser microfabrication contains the compound a. The photocurable resin composition contains, for example, a compound having polymerizability and a polymerization initiator apart from the compound a. Alternatively, the compound a functions as a polymerization initiator. The photocurable resin composition may further contain additives such as a binder resin. The photocurable resin composition may contain an epoxy resin.

The fluorescent microscope, for example, applies light to a biosample containing a fluorescent dye material and enables fluorescence emitted from the dye material to be observed. As an example, the fluorescent dye material to be added to the biosample contains the compound a.

### EXAMPLES

The following describes the present disclosure in more detail with reference to examples. The following examples are by way of example, and the present disclosure is not limited to the following examples.

### Example 1

A compound (2) represent by Formula (2) was synthesized by the following procedure.

First, 50 g (0.342 mol) of α-tetralone (manufactured by Tokyo Chemical Industry Co., Ltd.) and 500 mL of tetrahydrofuran anhydride (manufactured by FUJIFILM Wako Pure Chemical Corporation) were put into a reaction vessel with a volume of 2 L in an argon atmosphere. The obtained solution was cooled to −20° C., and 376 ml (0.376 mol) of sodium bis(trimethylsilyl)amide (a tetrahydrofuran solution with a concentration of 1.0 mol/L) (manufactured by FUJIFILM Wako Pure Chemical Corporation) was added dropwise thereto. The mixture was stirred for 15 minutes at −20° C., and then 23.4 ml (0.376 mol) of iodomethane (manufactured by FUJIFILM Wako Pure Chemical Corporation) was slowly added dropwise thereto. Subsequently, the temperature was raised to room temperature over 3 hours. The obtained suspension was added to 1.5 L of hydrochloric acid (0.5 mol/L) to divide it into two phases, and the aqueous phase was extracted with toluene. The organic phase was washed successively with an aqueous sodium bicarbonate solution, tap water, and 1 L of saturated saline solution and subjected to drying treatment using magnesium sulfate anhydride. Next, the extract was concentrated to obtain a pale brown liquid. The pale brown liquid was purified by evaporation to obtain a precursor of the compound (2) as a colorless liquid.

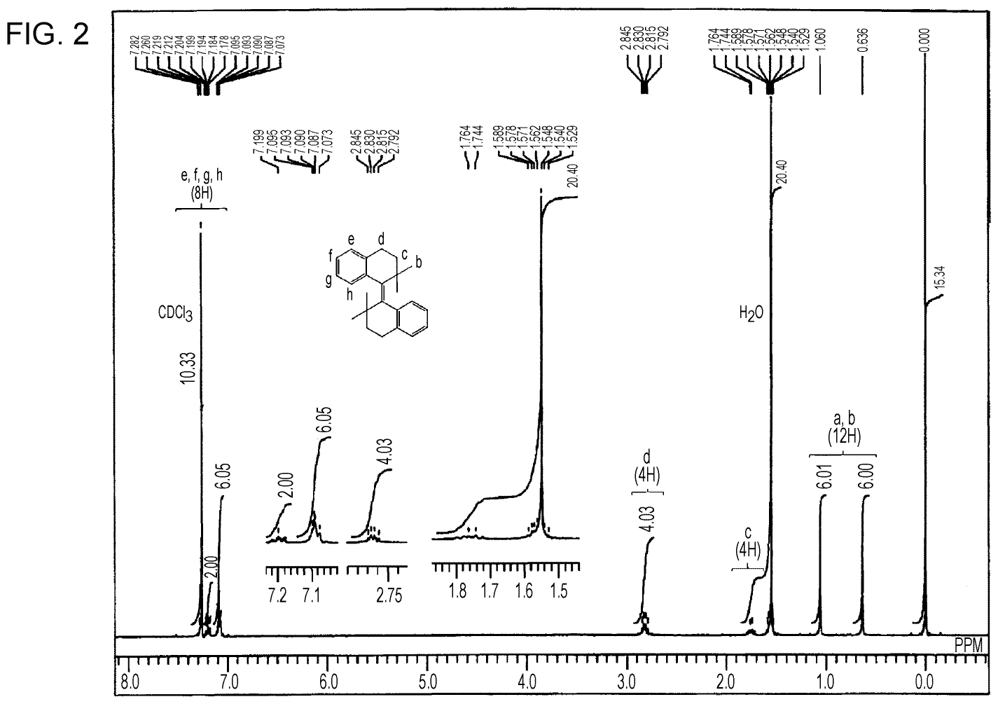

Next, 480 mL of tetrahydrofuran anhydride (manufactured by FUJIFILM Wako Pure Chemical Corporation) was put into a reaction vessel with a volume of 1 L in an argon atmosphere and cooled to −15° C., and then 11.0 mL (100 mmol) of titanium(IV) chloride (manufactured by FUJIFILM Wako Pure Chemical Corporation) was slowly added dropwise thereto. The mixture was stirred for 30 minutes at −15° C., then 19.7 g (301 mmol) of zinc powder (manufactured by Sigma-Aldrich) was added thereto at once, and the mixture was stirred for 30 minutes at −15° C. To the obtained bluish brown suspension, 12.0 g (66.9 mmol) of the precursor of the compound (2) diluted with 120 ml of tetrahydrofuran anhydride was added dropwise over 10 minutes. The cooling bath was removed, and the bath temperature was raised to 75° C., and the mixture was stirred for 30 minutes at 75° C. The obtained blackish brown suspension was added dropwise to a 2.0 mol/L aqueous solution of potassium carbonate (manufactured by FUJIFILM Wako Pure Chemical Corporation) (1 L), which was stirred for 1 hour at room temperature. The precipitated solid was subjected to celite filtration, and the filter bed was washed with 500 mL of ethyl acetate (manufactured by FUJIFILM Wako Pure Chemical Corporation). Out of the filtrate, the aqueous phase was extracted with 500 mL of a mixed liquid of heptane (manufactured by FUJIFILM Wako Pure Chemical Corporation)/ethyl acetate=1/1. The mixed liquid of the extract and the organic phase was washed successively with tap water and saturated saline solution and subjected to drying treatment using magnesium sulfate anhydride. The liquid obtained by the drying treatment was concentrated with a rotary evaporator. The obtained pale brown liquid was purified by silica gel column chromatography to obtain a pale yellow liquid. Ethanol (manufactured by FUJIFILM Wako Pure Chemical Corporation) in an amount of 12 mL was added to the pale yellow liquid, which was subjected to ultrasonic irradiation. The precipitated solid was filtered, and the filter bed was washed with methanol (manufactured by FUJIFILM Wako Pure Chemical Corporation) and dried to synthesize the compound (2) as a colorless powder. FIG. 2 is a graph showing a ¹H-NMR spectrum of the compound (2). The ¹H-NMR spectrum of the compound (2) was as follows:

1H-NMR (400 MHz, CHLOROFORM-D) 67.22-7.07 (m, 8H), 2.85-2.79 (m, 4H), 1.77-1.74 (m, 4H), 1.06 (s, 6H), 0.64 (s, 6H).

### Comparative Examples 1 to 4

Compounds of Comparative Examples 1 to 4 were prepared. The compounds of Comparative Examples 1 to 4 are represented by Formulae (7) to (10) below, respectively. The compounds of Formula (7) and Formula (9) were purchased from Sigma-Aldrich. The compounds of Formula (8) and Formula (10) were purchased from Tokyo Chemical Industry Co., Ltd.

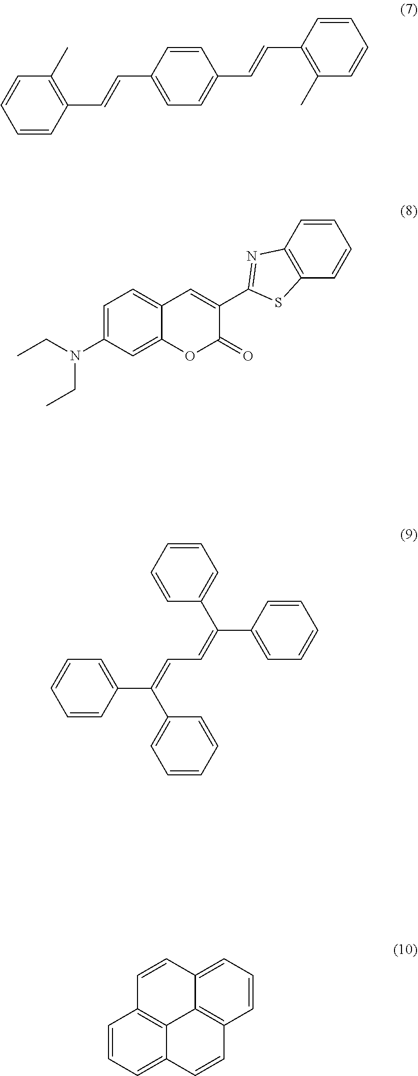

### Measurement of Two-Photon Absorption Cross Section

For the compounds of Example 1 and Comparative Examples 1 to 4, the two-photon absorption cross section against the light having a wavelength of 405 nm was measured. The measurement of the two-photon absorption cross section was performed using Z scan method described in J. Opt. Soc. Am. B, 2003, Vol. 20, p. 529. As a light source for measuring the two-photon absorption cross section, a titanium sapphire pulsed laser was used. Specifically, a second harmonic of the titanium sapphire pulsed laser was applied to a sample. The pulse width of the laser was 80 fs. The repetition frequency of the laser was 1 kHz. The average power of the laser was changed in a range of greater than or equal to 0.01 mW and less than or equal to 0.08 mW. The light from the laser was the light having a wavelength of 405 nm. Specifically, the light from the laser had a center wavelength of greater than or equal to 403 nm and less than or equal to 405 nm. The light from the laser had a full width at half maximum of 4 nm.

### Measurement of Molar Extinction Coefficient

For the compounds of Example 1 and Comparative Examples 1 to 4, the molar extinction coefficient was measured by a method conforming to the stipulation of JIS K0115: 2004. Specifically, first, a sample to be measured with a compound concentration adjusted to 500 mmol/L was prepared. For the sample to be measured, an absorption spectrum was measured. From the obtained spectrum, absorbance at a wavelength of 405 nm was read. Based on the compound concentration of the sample to be measured and the optical path length of a cell used for the measurement, the molar extinction coefficient was calculated.

Table 1 lists the two-photon absorption cross section a (GM) and the molar extinction coefficient ε(mol⁻¹·L·cm⁻¹) obtained by the methods described above. In Table 1, “SA” means that saturated absorption occurred during the two-photon absorption measurement by Z scan method, and the value of the cross section was not obtained.

TABLE 1

| | | | Molar |
|---|---|---|---|
| | | Two-photon | extinction |
| | | absorption | coefficient |
| | | cross section | (mol⁻¹ · L · |
| | Dye | (GM) | cm⁻¹) |
| Example 1 | Compound (2) | 15 | 160 |
| Comparative Example 1 | Compound (7) | SA | 470 |
| Comparative Example 2 | Compound (8) | SA | 17,000 |
| Comparative Example 3 | Compound (9) | SA | 210 |
| Comparative Example 4 | Compound (10) | 180 | 1 |

### Recording and Reproduction Characteristic

### Production of Thin Film Containing Dye

First, the following materials were mixed together by stirring to obtain a uniformly mixed application liquid. The weight ratio of the resin was fixed to 9 wt % with respect to the solvent. Although solubility to the solvent varied depending on the dyes, they were each dissolved to the upper limit of solubility. As the resin, poly(9-vinylcarbazole) (manufactured by Sigma-Aldrich) was used. As the solvent, chlorobenzene (manufactured by FUJIFILM Wako Pure Chemical Corporation) was used. Table 2 lists the composition ratios of application liquids for thin film production containing the compounds of Example 1 and Comparative Examples 1 to 4 as dyes.

TABLE 2

| | Dye | Resin | Solvent |
|---|---|---|---|
| Example 1 | Compound (2) | 9.0 parts by | 91.0 parts by |
| | 8.6 parts by mass | mass | mass |
| Comparative | Compound (7) | 9.0 parts by | 91.0 parts by |
| Example 1 | 14.1 parts by mass | mass | mass |
| Comparative | Compound (8) | 9.0 parts by | 91.0 parts by |
| Example 2 | 9.6 parts by mass | mass | mass |
| Comparative | Compound (9) | 9.0 parts by | 91.0 parts by |
| Example 3 | 24.4 parts by mass | mass | mass |
| Comparative | Compound (10) | 9.0 parts by | 91.0 parts by |
| Example 4 | 18.4 parts by mass | mass | mass |

Next, a glass substrate was prepared. The dimensions of the glass substrate were 26 mm long, 38 mm wide, and 0.9 mm thick. The glass substrate was set in a spin coater. The application liquid produced by the above method in an amount of 400 L was dropped onto the glass substrate, which was rotated for 30 seconds at a number of revolutions of 3,000 rpm. Subsequently, the glass substrate was dried on a hot plate at 80° C. for 30 minutes to obtain a resin thin film containing any compound of Example 1 and Comparative Examples 1 to 4. In the following, these resin thin films will be referred to as a thin film of Example 1 and thin films of Comparative Examples 1 to 4.

Reproduction Operation before Recording

Pulsed light with a center wavelength of 405 nm, a peak power of 3 mW, a pulse width of 200 nanoseconds, and a repetition frequency of 100 Hz was applied through a lens with an NA of 0.85 so as to be focused on the resin thin film on the glass substrate. Reflected light signal intensity at this time was acquired as initial reflected light signal intensity. It was determined that a recording mark was formed when the reflected light signal intensity after a recording operation changed with respect to the initial reflected light signal intensity.

### Recording Operation

One pulse of recording light with a center wavelength of 405 nm and a peak power of 100 mW was applied through a lens with an NA of 0.85 to perform recording. The pulse width was adjusted from 10 nanoseconds to 5 milliseconds.

### Reproduction Operation

Light with a center wavelength of 405 nm, a peak power of 3 mW, and a pulse width of 200 nanoseconds was applied with a repetition frequency of 100 Hz through a lens with an NA of 0.85 to a recording portion of the resin thin film to acquire reflected light signal intensity. A change rate of the reflected light signal intensity after the recording operation with respect to the reflected light signal intensity before the recording operation was calculated.

### Evaluation of Recording and Reproduction Characteristic

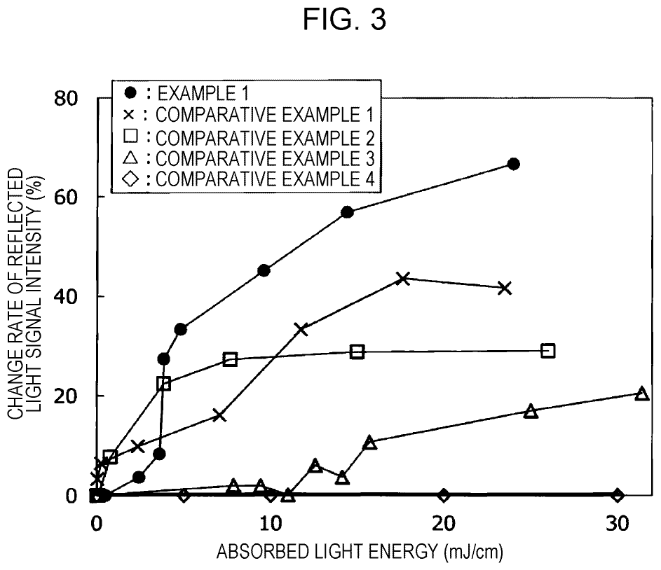

Because the dyes differ in the molar extinction coefficient and solubility, the produced resin thin films have different optical absorption characteristics. To perform an equal comparison of the recording and reproduction characteristic, incident light intensity at the time of recording was converted into the energy (J/cm) of light with a wavelength of 405 nm absorbed by the resin thin film of 1 cm thick. The energy (J/cm) of the light absorbed by the resin thin film of 1 cm thick was calculated by multiplying the intensity (W) of the applied light by a recording time (seconds) and the absorption coefficient (cm⁻¹) of the resin thin film. The absorption coefficient of the resin thin film was calculated by multiplying a dye concentration (mol/L) in the thin film by the molar extinction coefficient (mol⁻¹·L·cm⁻¹) of the dye. FIG. 3 shows a graph plotting the change rate of the reflected light signal intensity against the energy of the light absorbed by the resin thin film of 1 cm thick.

FIG. 3 is a graph showing the recording and reproduction characteristic of the resin thin films of the example and the comparative examples. In FIG. 3, the horizontal axis represents absorbed light energy changed by the application time (the pulse width) of the laser light. The vertical axis represents the change rate of the reflected light signal intensity after the recording operation with respect to the reflected light signal intensity before the recording operation. In FIG. 3, the change in the reflected light signal intensity being small means that the resin thin film hardly altered even by applying the laser light. The change in the reflected light signal intensity being large means that the resin thin film altered by the application of the laser light to form the recording mark.

As can be seen from FIG. 3, in Example 1, when the application time (the pulse width) of the laser light was increased to increase the absorbed light energy, the change rate of the reflected light signal intensity suddenly increased near 3 mJ/cm. That is, the thin film of Example 1 had a threshold characteristic in which the reflection light signal intensity hardly changed even when low-intensity light was applied, whereas the reflected light signal intensity significantly changed by applying high-intensity light. That is, the thin film of Example 1 hardly altered even when the reproduction operation was repeated and thus had high durability and high reliability. When the thin film is hard to alter by the weak light during reproduction, formation of the recording mark despite not performing the recording operation can be avoided.

Further, in Example 1, the change rate of the reflected light signal intensity was hard to be saturated to increase up to greater than or equal to 60%. The higher the change rate of the reflected light signal intensity, the larger the difference between the reflected light signal intensity of the recording mark and the reflected light signal intensity of the surroundings of the recording mark. That is, an S/N ratio, which is the ratio of signal to noise, improves, making it easy to read the recording mark.

The threshold characteristic of the thin film of Example 1 represents that non-linear optical absorption, or specifically, excited state absorption conspicuously occurred in a range in which the absorbed light energy was greater than 3 mJ/cm.

The compound (2) of Example 1 has a structure in which tetralin rings are coupled to each other with a carbon-carbon double bond. It is estimated that this structure caused a good result in the recording and reproduction characteristic. Alternatively, the compound (2) can also be regarded as being a compound having a structure in which benzen rings of trans-stilbene and double-bond carbons are bound together by alkyl chains. It is considered that such a structure improves the isomerization rate of the compound and has an influence on the improvement in the recording and reproduction characteristic.

On the other hand, in Comparative Examples 1 to 4, the change rate of the reflected light signal intensity increased linearly against the absorbed light energy and was saturated with a low change rate. In addition, in Comparative Example 1 and Comparative Example 2, even when the absorbed light energy was less than or equal to 1 mJ/cm, changes in the reflected light signal intensity of about 8% to 10% occurred. This means that the resin thin film easily alters by repeating the reproduction operation. That is, the thin film altering by the weak light during reproduction means that the recording mark is formed despite not performing the recording operation.

In Comparative Example 3, the change rate of the reflected light signal intensity changed linearly against the absorbed light energy and, in addition, had a small change rate of the reflected light signal intensity against the absorbed light energy. This means that the S/N ratio is low, making it hard to read the presence or absence of the recording mark.

In the case of the thin film of Comparative Example 4, no change in the reflected light signal intensity occurred in the range of light intensity used in the experiment.

The non-linear optical-absorbing material of the present disclosure can be used for uses such as recording layers of three-dimensional optical memories and photocurable resin compositions for three-dimensional (3D) laser microfabrication. The non-linear optical-absorbing material of the present disclosure has an optical absorption characteristic showing high non-linearity against light having a wavelength in a short-wavelength range. Thus, the non-linear optical-absorbing material of the present disclosure can achieve extremely high spatial resolution in uses such as three-dimensional optical memories and shaping machines. Compared to conventional non-linear optical-absorbing materials, the non-linear optical-absorbing material of the present disclosure can cause non-linear optical absorption preferentially over one-photon absorption even when laser light with low light intensity is applied.

What is claimed is:

1. A non-linear optical-absorbing material comprising a compound represented by Formula (1) below: 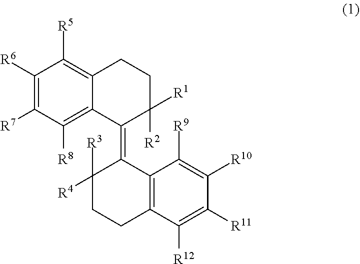
in Formula (1) above, R¹ to R¹² mutually independently represent a group containing at least one atom selected from the group consisting of H, B, C, N, O, F, Si, P, S, Cl, I, and Br.

2. The non-linear optical-absorbing material according to claim 1, wherein R⁵ to R¹² are each a hydrogen atom.

3. The non-linear optical-absorbing material according to claim 1, wherein R¹ and R² are a same group or R¹ and R³ are a same group.

4. The non-linear optical-absorbing material according to claim 1, wherein R¹ to R⁴ are a same group.

5. The non-linear optical-absorbing material according to claim 1, wherein R¹ to R⁴ are each a methyl group.

6. The non-linear optical-absorbing material according to claim 1, wherein the non-linear light-absorbing material is used for a device using light having a wavelength of longer than or equal to 390 nm and shorter than or equal to 420 nm.

7. A recording medium comprising the non-linear optical-absorbing material according to claim 1.

8. A method for recording information, the method comprising:
preparing a light source emitting light having a wavelength of longer than or equal to 390 nm and shorter than or equal to 420 nm; and
concentrating the light from the light source and applying the light to a recording layer of a recording medium containing the non-linear optical-absorbing material according to claim 1.

9. A method for reading information recorded by the method for recording information according to claim 8, the method for reading information comprising:
applying light to the recording layer of the recording medium to measure an optical characteristic of the recording layer; and
reading information from the recording layer.

10. The method for reading information according to claim 9, wherein the optical characteristic is intensity of light reflected by the recording layer.
